(19) United States
(12) Patent Application Publication
(10) Pub. No.: US 2021/0093203 A1
(43) Pub. Date: Apr. 1, 2021

(54) SYSTEMS AND METHODS OF DETERMINING HEART-RATE AND RESPIRATORY RATE FROM A RADAR SIGNAL USING MACHINE LEARNING METHODS

(71) Applicant: DawnLight Technologies, Palo Alto, CA (US)

(72) Inventors: Erheng ZHONG, Palo Alto, CA (US); YuJia LI, Palo Alto, CA (US); Nan LIU, Palo Alto, CA (US); Ke ZHAI, Palo Alto, CA (US)

(21) Appl. No.: 16/588,443

(22) Filed: Sep. 30, 2019

(51) Int. Cl. A61B 5/024; A61B 5/05; A61B 5/00; G06N 3/08; G06N 20/00

(52) U.S. Cl. CPC A61B 5/024; A61B 5/05; G06N 20/00; A61B 5/7225; G06N 3/084; A61B 5/7203

(57) ABSTRACT

A computer-implemented method for determining a heart rate and respiratory rate from a radio frequency signal comprises inputting a radio frequency signal obtained from a test subject into a neural network. The method further comprises training the neural network using the radio frequency signal and extracting a heart rate and a respiratory rate from the radio frequency signal using the neural network. Further, the method comprises comparing the heart rate and the respiratory rate extracted from the radio frequency signal to a verifiable heart rate and verifiable respiratory rate for the test subject to compute an error measure. Finally, the method comprises using the error measure to apply back propagation to adjust front end parameters for one or more layers of the neural networks to improve a prediction accuracy of the neural network.

### FIELD OF THE INVENTION

Embodiments according to the present invention relate to a non-contact sensing system configured to detect a heart rate and respiration rate of a user.

### BACKGROUND OF THE INVENTION

In recent years, there has been increasing interest in contactless vital signs monitoring. The technology related to contactless and non-invasive monitoring of vital signs may find widespread adoption in connection with several applications including clinical care, home health-care, airport screening, and automated driving systems. Furthermore, the technology can also be widely used for disaster relief (e.g., to determine if victims of disaster are alive), in connection with severe burn patients and patients with infectious diseases, for clinical dynamic monitoring of infants and the elderly, as well as in connection with monitoring sleep quality. Monitoring physiological parameters in modern medical tests, for example, may also provide a reliable and important basis for doctors to diagnose and treat various health conditions afflicting a patient.

Contactless monitoring of vital signs, such as heart rate and respiratory rate, is significantly more pragmatic than requiring a user to wear a device such as a heart rate monitor. Such devices may be intrusive to the user, and are inconvenient to wear on an everyday basis. Further, it can be difficult to determine whether changes in heart rate, heart rate variability, and the like are attributable to stress or other physiological conditions, or unrelated factors such as the user's movement and activity.

One of the ways in which vital signs may be monitored wirelessly is by using radar technology. Radar uses operating principle in which, when radio energy (a short pulse) is emitted from a directional antenna and collides against a target object, waves are reflected, that is, part of the energy returns, and the direction of the target object can be detected using a device for receiving and detecting a reflected wave. In other words, radar is equipment for transmitting a radio wave to a target object, receiving the reflected waves of the energy of the radio waves, and measuring the position (direction and distance) of the target object using the round-trip time and the directional characteristics of an antenna based on the straightness and isochronism of a radio wave.

In particular, using Doppler radar technology for monitoring vital signs has been an increasingly active field of research. The Doppler shifts caused by the mechanical movements of the heart and the lungs can be detected and analyzed to determine the heart rate and the respiration rate. A continuous-wave (CW) radar (also known as a Doppler radar) transmits a radio frequency single-tone continuous-wave signal which is reflected by a target and then demodulated in a receiver. By the Doppler effect, the radio frequency signal reflected by the moving tissue of the target undergoes a frequency shift proportional to the surface velocity of the tissue. If the moving tissue has a periodic motion (as the tissue in the chest region of a subject may have due to the periodic motion of the heart and the lungs) the Doppler effect results in a phase shift of the reflected radio frequency signal which is proportional to the instantaneous surface displacement. In the receiver, the transmitted signal may be mixed with the reflected Doppler-shifted signal to produce a mixing product which, following low pass filtering, results in a baseband signal including a low frequency component that is directly proportional to the instantaneous surface displacement.

One of the challenges of using Doppler radar technology for detecting vital signs such as heart rate and respiratory rate is the extraction of the low frequency component from the baseband signal, in particular, because the maximum amplitudes of the chest region displacements due to the heart beat and the respiration are much smaller than the wavelength of the radio frequency signal. Random movements of a subject further exacerbate this problem. In case the subject moves randomly during measurement, thereby, causing a random displacement of the reflecting tissue, reliable extraction of the heartbeat and respiration rates from the baseband signal can be severely hampered.

One of the most significant drawbacks of conventional methods of using continuous-wave radar systems to monitor vital signs is that none of the existing technologies manage to adequately solve the problem of accurately accounting for random physical movements by the test subject. Further, conventional non-contact methods of monitoring vital signs are not sufficiently accurate precisely because they cannot reliably distinguish between chest region displacements due to heartbeat and respiration from displacement caused by other factors such as random subject movement.

### BRIEF SUMMARY OF THE INVENTION

Accordingly, a need exists for a non-contact vital signs detection system that can address the problems with the systems described above. Using the beneficial aspects of the systems described, without their respective limitations, embodiments of the present invention provide a novel solution to address these problems.

Embodiments of the present invention enable contactless detection of at least one of a heart rate and a respiratory rate of a subject using machine learning methods, which can be trained to be less sensitive to random movements of the subject. Machine learning is the umbrella term for computational techniques that allows models learn from data rather than following strict programming rules. Machine learning algorithms build a mathematical model based on sample data, known as “training data,” in order to make predictions or decisions without being explicitly programmed to perform the task. Machine learning includes using several different types of models including artificial neural networks (ANNs), deep learning methods, etc.

Artificial neural networks (ANN) are computing systems that are inspired by, but not identical to, biological neural networks that constitute animal brains. Such systems “learn” to perform tasks by considering examples, generally without being programmed with task-specific rules. Other types of neural networks include recurrent neural networks (RNN), convolutional neural networks (CNNs), deep belief networks, etc. Some neural networks comprise multiple layers that enable hierarchical feature learning.

Deep learning (also known as deep structured learning or hierarchical learning) is part of the broader family of machine learning methods based on ANNs. Deep learning describes learning that includes learning hierarchical features from raw input data and leveraging such learned features to make predictions associated with the raw input data.

In particular, embodiments of the present invention train machine learning models, e.g., an artificial neural network to predict the heart-rate and respiratory rate by collecting and using measurements (including, for example, actual heart rate measurements from an electrocardiogram (EKG) monitor) from a variety of test subjects using machine-learning methods. Having trained the machine learning model, embodiments of the present invention can use the model to predict the heart-rate and respiratory rate for subjects accurately (without needing additional data from, for example, an EKG monitor). By using a machine learning model to train over several test subjects, each with their own unique movements, embodiments of the present invention are able to provide significantly more accurate results for new subjects. The trained machine learning model is able to account for random subject movements based on information cognized through the training process.

In one embodiment, a computer-implemented method for determining a heart rate from a radio frequency signal is disclosed. The method comprises inputting a first radio frequency signal obtained from a first test subject into a machine learning model, wherein the first radio frequency signal is comprised within a training set for training the machine learning model. The method further comprises training the machine learning model using the first radio frequency signal and extracting a first heart rate and a first respiratory rate from the first radio frequency signal using the machine learning model. Thereafter, the method comprises comparing the first heart rate and the first respiratory rate extracted from the first radio frequency signal to a verifiable heart rate and verifiable respiratory rate for the first test subject to compute an error measure. Additionally, the method comprises using the error measure to apply back propagation to adjust front end parameters for one or more layers of the machine learning model to improve a prediction accuracy of the machine learning model. In one embodiment, the method comprises applying the machine learning model to a second radio frequency signal obtained from a second test subject to predict a second heart rate and second respiratory rate for the second test subject, wherein values of the second heart rate and the second respiratory rate are more accurate than the first heart rate and the first respiratory rate.

In another embodiment, a non-transitory computer-readable storage medium having stored thereon, computer executable instructions that, if executed by a computer system cause the computer system to perform a method for determining a heart rate from a wireless signal is disclosed. The method comprises inputting a first wireless signal obtained from a first test subject into a machine learning model, wherein the first wireless signal is comprised within a training set for training the machine learning model. Further, the method comprises training the machine learning model using the first wireless signal and extracting a first heart rate from the first wireless signal using the machine learning model. Further, the method comprises comparing the first heart rate extracted from the wireless signal to a verifiable heart rate for the first test subject to compute an error measure and using the error measure to apply back propagation to adjust front end parameters for one or more layers of the machine learning model to improve a prediction accuracy of the machine learning model. In one embodiment, the method also comprises applying the machine learning model to a second wireless signal obtained from a second test subject to predict a second heart rate for the second test subject, wherein values of the second heart rate are more accurate than the first heart rate and the first respiratory rate.

In a different embodiment, a system for determining a respiratory rate from a radio frequency signal is disclosed. The system comprises a memory for storing a time-domain representation of one or more radio frequency signals, instructions associated with a neural network and a process of determining the respiratory rate from the radio frequency signal. Further the system comprises a processor coupled to the memory, the processor configured to operate in accordance with the instructions to: (a) input a first radio frequency signal obtained from a first test subject into the neural network, wherein the first radio frequency signal is comprised within a training set for training the neural network; (b) train the neural network using the first radio frequency signal; (c) extract a first respiratory rate from the first radio frequency signal using the neural network; (d) compare the first heart rate extracted from the first radio frequency signal to a verifiable respiratory rate for the first test subject to compute an error measure; and (e) use the error measure to apply back propagation to adjust front end parameters for one or more layers of the neural network to improve a prediction accuracy of the neural network.

The following detailed description together with the accompanying drawings will provide a better understanding of the nature and advantages of the present invention.

### BRIEF DESCRIPTION OF THE DRAWINGS

Embodiments of the present invention are illustrated by way of example, and not by way of limitation, in the figures of the accompanying drawings and in which like reference numerals refer to similar elements.

FIG. 1 is an exemplary computer system in accordance with embodiments of the present invention.

FIG. 2 illustrates the time-domain and frequency domain characteristics of a linear FM-CW chirp sequence.

FIG. 3 illustrates the manner in which a mixed transmitted and received radar signal, comprising heart-rate and respiratory rate information, can be converted into a time domain signal for further processing and vital signs estimation in accordance with an embodiment of the present invention.

FIG. 4 illustrates a conventional spectrum based method for determining breath rate and heart-rate.

FIG. 5 illustrates a high-level flow diagram providing an overview of the manner in which heart-rate and respiratory rate may be detected in accordance with an embodiment of the present invention.

FIG. 6 is a flow diagram indicating the manner in which a machine learning model such as a neural network may be trained to perform contactless detection of at least one of a heart rate and a respiratory rate of a subject using deep-learning methods in accordance with an embodiment of the present invention.

FIG. 7 illustrates an exemplary method in which binning may be performed for the signal waveform containing respiratory rate and heart-rate information in accordance with an embodiment of the present invention.

FIG. 8 is a flow diagram indicating the manner in which a machine learning model such as a neural network may be used to perform contactless prediction of a heart-rate and respiratory rate using deep-learning methods in accordance with an embodiment of the present invention.

FIG. 9 illustrates an exemplary apparatus for performing contactless prediction of a heart-rate and respiratory rate using a machine learning model in accordance with an embodiment of the present invention.

FIG. 10 illustrates the manner in which a long sliding time window may be maintained in conjunction with a short sliding window in order to detect sudden changes in heart-rate in accordance with an embodiment of the present invention.

FIG. 11 depicts a flowchart illustrating an exemplary computer-implemented process for training and using a machine learning model to detect respiration rate and heart rate in accordance with an embodiment of the present invention.

FIG. 12 depicts a flowchart illustrating an exemplary computer-implemented process for using a machine learning model to detect sudden changes in heart rate in accordance with an embodiment of the present invention.

In the figures, elements having the same designation have the same or similar function.

### DETAILED DESCRIPTION OF THE INVENTION

Reference will now be made in detail to embodiments, examples of which are illustrated in the accompanying drawings. While the embodiments will be described in conjunction with the drawings, it will be understood that they are not intended to limit the embodiments. On the contrary, the embodiments are intended to cover alternatives, modifications and equivalents. Furthermore, in the following detailed description, numerous specific details are set forth in order to provide a thorough understanding. However, it will be recognized by one of ordinary skill in the art that the embodiments may be practiced without these specific details. In other instances, well-known methods, procedures, components, and circuits have not been described in detail as not to unnecessarily obscure aspects of the embodiments.

### Notation and Nomenclature Section

Some regions of the detailed descriptions which follow are presented in terms of procedures, logic blocks, processing and other symbolic representations of operations on data bits within a computer memory. These descriptions and representations are the means used by those skilled in the data processing arts to most effectively convey the substance of their work to others skilled in the art. In the present application, a procedure, logic block, process, or the like, is conceived to be a self-consistent sequence of steps or instructions leading to a desired result. The steps are those requiring physical manipulations of physical quantities. Usually, although not necessarily, these quantities take the form of electrical or magnetic signals capable of being stored, transferred, combined, compared, and otherwise manipulated in a computer system.

It should be borne in mind, however, that all of these and similar terms are to be associated with the appropriate physical quantities and are merely convenient labels applied to these quantities. Unless specifically stated otherwise as apparent from the following discussions, it is appreciated that throughout the present invention, discussions utilizing the terms such as “generating,” “extracting,” “sampling,” “inputting,” “training,” “comparing,” “performing,” “using,” “applying,” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

The description below provides a discussion of computers and other devices that may include one or more modules. As used herein, the term “module” or “block” may be understood to refer to software, firmware, hardware, and/or various combinations thereof. It is noted that the blocks and modules are exemplary. The blocks or modules may be combined, integrated, separated, and/or duplicated to support various applications. Also, a function described herein as being performed at a particular module or block may be performed at one or more other modules or blocks and/or by one or more other devices instead of or in addition to the function performed at the described particular module or block. Further, the modules or blocks may be implemented across multiple devices and/or other components local or remote to one another. Additionally, the modules or blocks may be moved from one device and added to another device, and/or may be included in both devices. Any software implementations of the present invention may be tangibly embodied in one or more storage media, such as, for example, a memory device, a floppy disk, a compact disk (CD), a digital versatile disk (DVD), or other devices that may store computer code.

The terminology used herein is for the purpose of describing particular embodiments only and is not intended to limit the scope of the present invention. As used throughout this disclosure, the singular forms “a,” “an,” and “the” include plural reference unless the context clearly dictates otherwise. Thus, for example, a reference to “a module” includes a plurality of such modules, as well as a single module, and equivalents thereof known to those skilled in the art.

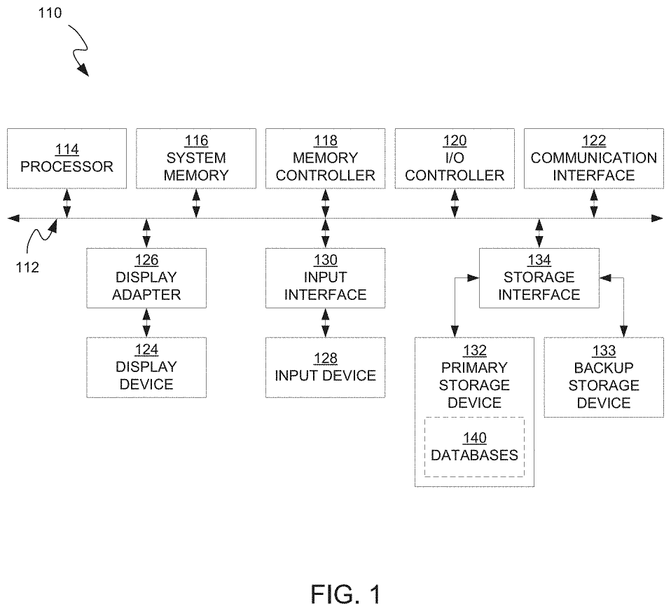

FIG. 1 is a block diagram of an example of a computing system 110 used to determine respiratory rate and heart-rate from a radar signal and capable of implementing embodiments of the present disclosure. Computing system 110 broadly represents any single or multi-processor computing device or system capable of executing computer-readable instructions. Examples of computing system 110 include, without limitation, workstations, laptops, client-side terminals, servers, distributed computing systems, handheld devices, or any other computing system or device. In its most basic configuration, computing system 110 may include at least one processor 114 and a system memory 116.

Processor 114 generally represents any type or form of processing unit capable of processing data or interpreting and executing instructions. In certain embodiments, processor 114 may receive instructions from a software application or module. These instructions may cause processor 114 to perform the functions of one or more of the example embodiments described and/or illustrated herein.

System memory 116 generally represents any type or form of volatile or non-volatile storage device or medium capable of storing data and/or other computer-readable instructions. Examples of system memory 116 include, without limitation, RAM, ROM, flash memory, or any other suitable memory device. Although not required, in certain embodiments computing system 110 may include both a volatile memory unit (such as, for example, system memory 116) and a non-volatile storage device (such as, for example, primary storage device 132).

Computing system 110 may also include one or more components or elements in addition to processor 114 and system memory 116. For example, in the embodiment of FIG. 1, computing system 110 includes a memory controller 118, an input/output (I/O) controller 120, and a communication interface 122, each of which may be interconnected via a communication infrastructure 112. Communication infrastructure 112 generally represents any type or form of infrastructure capable of facilitating communication between one or more components of a computing device. Examples of communication infrastructure 112 include, without limitation, a communication bus (such as an Industry Standard Architecture (ISA), Peripheral Component Interconnect (PCI), PCI Express (PCIe), or similar bus) and a network.

Memory controller 118 generally represents any type or form of device capable of handling memory or data or controlling communication between one or more components of computing system 110. For example, memory controller 118 may control communication between processor 114, system memory 116, and 1/O controller 120 via communication infrastructure 112.

I/O controller 120 generally represents any type or form of module capable of coordinating and/or controlling the input and output functions of a computing device. For example, I/O controller 120 may control or facilitate transfer of data between one or more elements of computing system 110, such as processor 114, system memory 116, communication interface 122, display adapter 126, input interface 130, and storage interface 134.

Communication interface 122 broadly represents any type or form of communication device or adapter capable of facilitating communication between example computing system 110 and one or more additional devices. For example, communication interface 122 may facilitate communication between computing system 110 and a private or public network including additional computing systems. Examples of communication interface 122 include, without limitation, a wired network interface (such as a network interface card), a wireless network interface (such as a wireless network interface card), a modem, and any other suitable interface. In one embodiment, communication interface 122 provides a direct connection to a remote server via a direct link to a network, such as the Internet. Communication interface 122 may also indirectly provide such a connection through any other suitable connection.

Communication interface 122 may also represent a host adapter configured to facilitate communication between computing system 110 and one or more additional network or storage devices via an external bus or communications channel. Examples of host adapters include, without limitation. Small Computer System Interface (SCSI) host adapters, Universal Serial Bus (USB) host adapters, IEEE (Institute of Electrical and Electronics Engineers) 1394 host adapters, Serial Advanced Technology Attachment (SATA) and External SATA (eSATA) host adapters, Advanced Technology Attachment (ATA) and Parallel ATA (PATA) host adapters, Fibre Channel interface adapters, Ethernet adapters, or the like. Communication interface 122 may also allow computing system 110 to engage in distributed or remote computing. For example, communication interface 122 may receive instructions from a remote device or send instructions to a remote device for execution.

As illustrated in FIG. 1, computing system 110 may also include at least one display device 124 coupled to communication infrastructure 112 via a display adapter 126. Display device 124 generally represents any type or form of device capable of visually displaying information forwarded by display adapter 126. Similarly, display adapter 126 generally represents any type or form of device configured to forward graphics, text, and other data for display on display device 124.

As illustrated in FIG. 1, computing system 110 may also include at least one input device 128 coupled to communication infrastructure 112 via an input interface 130. Input device 128 generally represents any type or form of input device capable of providing input, either computer- or human-generated, to computing system 110. Examples of input device 128 include, without limitation, a keyboard, a pointing device, a speech recognition device, or any other input device.

As illustrated in FIG. 1, computing system 110 may also include a primary storage device 132 and a backup storage device 133 coupled to communication infrastructure 112 via a storage interface 134. Storage devices 132 and 133 generally represent any type or form of storage device or medium capable of storing data and/or other computer-readable instructions. For example, storage devices 132 and 133 may be a magnetic disk drive (e.g., a so-called hard drive), a floppy disk drive, a magnetic tape drive, an optical disk drive, a flash drive, or the like. Storage interface 134 generally represents any type or form of interface or device for transferring data between storage devices 132 and 133 and other components of computing system 110.

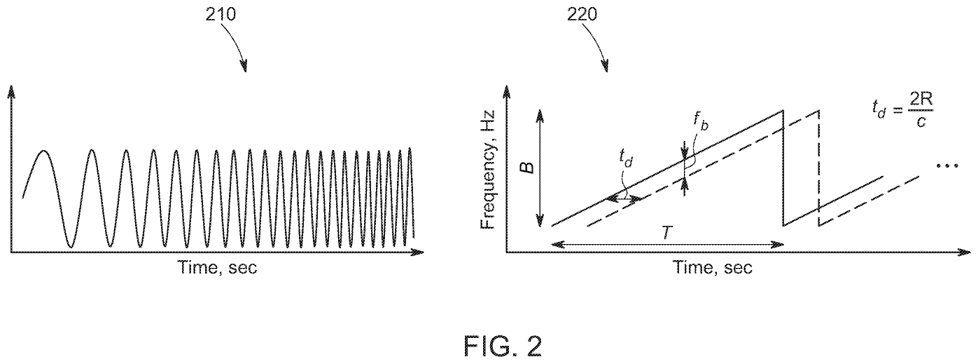

In one example, databases 140 may be stored in primary storage device 132. Databases 140 may represent portions of a single database or computing device or it may represent multiple databases or computing devices. For example, databases 140 may represent (be stored on) a portion of computing system 110 and/or portions of example network architecture 200 in FIG. 2 (below). Alternatively, databases 140 may represent (be stored on) one or more physically separate devices capable of being accessed by a computing device, such as computing system 110 and/or portions of network architecture 200.

Continuing with reference to FIG. 1, storage devices 132 and 133 may be configured to read from and/or write to a removable storage unit configured to store computer software, data, or other computer-readable information. Examples of suitable removable storage units include, without limitation, a floppy disk, a magnetic tape, an optical disk, a flash memory device, or the like. Storage devices 132 and 133 may also include other similar structures or devices for allowing computer software, data, or other computer-readable instructions to be loaded into computing system 110. For example, storage devices 132 and 133 may be configured to read and write software, data, or other computer-readable information. Storage devices 132 and 133 may also be a part of computing system 110 or may be separate devices accessed through other interface systems.

Many other devices or subsystems may be connected to computing system 110. Conversely, all of the components and devices illustrated in FIG. 1 need not be present to practice the embodiments described herein. The devices and subsystems referenced above may also be interconnected in different ways from that shown in FIG. 1. Computing system 110 may also employ any number of software, firmware, and/or hardware configurations. For example, the example embodiments disclosed herein may be encoded as a computer program (also referred to as computer software, software applications, computer-readable instructions, or computer control logic) on a computer-readable medium.

The computer-readable medium containing the computer program may be loaded into computing system 110. All or a portion of the computer program stored on the computer-readable medium may then be stored in system memory 116 and/or various portions of storage devices 132 and 133. When executed by processor 114, a computer program loaded into computing system 110 may cause processor 114 to perform and/or be a means for performing the functions of the example embodiments described and/or illustrated herein. Additionally or alternatively, the example embodiments described and/or illustrated herein may be implemented in firmware and/or hardware.

Systems and Methods of Determining Heart-Rate and Respiratory Rate from a Radar Signal Using Deep Learning Methods

One of the ways in which vital signs may be monitored wirelessly is by using radar technology. Continuous-wave radar is a type of radar system where a known stable frequency of continuous wave radio energy is transmitted and then received from any reflecting objects. Continuous-wave (CW) radar uses Doppler, which renders the radar immune to interference from large stationary objects and slow moving clutter. Frequency-modulated continuous-wave radar (FM-CW)— also called continuous-wave frequency-modulated (CWFM) radar—is a short-range measuring radar set capable of determining distance. This increases reliability by providing distance measurement along with speed measurement, which is essential when there is more than one source of reflection arriving at the radar antenna.

It is appreciated that FM-CW radar may be used to perform contactless monitoring of vital signs such as heart-rate and respiration. The radio frequency signal may be transmitted, for example, towards a chest region of the subject. The reflected signal may accordingly be Doppler-shifted due to tissue displacement in the chest region caused by at least one of the heart rate and the respiratory rate. The displaced tissue reflecting the transmitted signal may include any one, or a combination of, the chest wall, the heart and the lungs of the subject. The reflected signal may be demodulated in a receiver and analyzed to determine the heart rate and/or the respiratory rate.

By the Doppler effect, the radio frequency signal reflected by the moving tissue of the target undergoes a frequency shift proportional to the surface velocity of the tissue. If the moving tissue has a periodic motion (as the tissue in the chest region of a subject may have due to the periodic motion of the heart and the lungs) the Doppler effect results in a phase shift of the reflected radio frequency signal which is proportional to the instantaneous surface displacement. In the receiver, the transmitted signal may be mixed with the reflected Doppler-shifted signal to produce a mixing product which, following low pass filtering, results in a baseband signal including a low frequency component that is directly proportional to the instantaneous surface displacement.

FIG. 2 illustrates the time-domain and frequency domain characteristics of a linear FM-CW chirp sequence. Plot 210 shows the time-domain characteristics of the linear FM-CW chirp sequence. Plot 220, meanwhile, shows the frequency versus time characteristics of the chirp sequence. The transmitted signal may be a linear FM-CW chirp sequence, the frequency and time-domain characteristics of which follow a saw-tooth pattern as shown in plot 220.

The transmitted FM-CW signal may be indicated by relation (1) below.

$$s ( t ) = e {\text{?}}^{j ( 2 π f + π \frac{B}{T} )} \text{?} \\ \text{?} \text{indicates text missing or illegible when filed} \qquad ( 1 )$$

The signal at the receiver may be represented as a delayed version of transmitted signal given by relation (2) below.

$$r ( t ) = e {\text{?}}^{j ( 2 π f ( t - {t}_{d} ) + π \frac{B}{T} {( t - {t}_{d} )}^{2} )} \\ \text{?} \text{indicates text missing or illegible when filed} \qquad ( 2 )$$

By mixing the transmitted and received signals, the below relation (3) is obtained.

$$s ( t ) \cdot r ( t ) \approx {e}^{j ( 4 π \frac{BR}{cT} t + \frac{4 π}{λ} R )} = {e}^{j ( {f}_{b} t + {φ}_{b} )} \qquad ( 3 )$$

In relation (3) above, frequency, f_{b} is directly correlated to the distance between the object and the radar (e.g., the radar receiver or antenna), whereas ϕ_{b} is closely related to the velocity of the object. Both f_{b} and ϕ_{b} can be calculated by applying a Fast Fourier Transform (FFT) on the mixed signal. Specifically, to determine vital signs, f_{b} provides the distance between the subject and the radar and is used to determine the range bins (reflecting distance) of the test subjects while ϕ_{b} reflects the velocity and/or displacement of the subject's chest.

The FFT can be applied to the mixed signal represented by relation (3) for each chirp to obtain a range profile, which represents the reflection signal strength in each range bin. The range bin with the highest signal strength can then be selected. Thereafter, the phase of the selected range bin can be calculated using an arctan(imaginary, real) function, which constructs the waveform in the time domain taking into account multiple chirps.

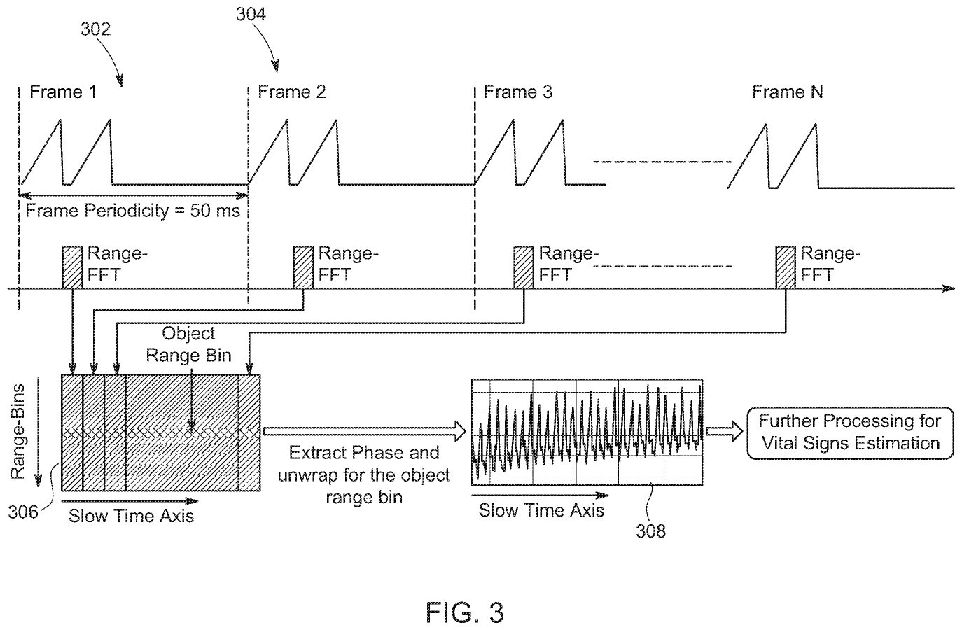

FIG. 3 illustrates the manner in which a mixed transmitted and received radar signal, comprising heart-rate and respiratory rate information, can be converted into a time domain signal for further processing and vital signs estimation in accordance with an embodiment of the present invention. As shown in FIG. 3, an FFT is applied to each chirp (e.g. chirp 302 in Frame 1, chirp 304 in Frame 2) to obtain a range profile 306, e.g., stored in computer readable memory. The range bin with the highest signal strength can then be selected from range profile 306 and the phase can be extracted to prepare a time domain signal 308 (in slow time) for further processing and vital signs estimation.

As mentioned previously, one of the significant drawbacks of conventional methods, e.g., spectrum methods of using continuous-wave radar systems to monitor vital signs is that none of the existing technologies manage to adequately solve the problem of accurately accounting for random physical movements by the test subject.

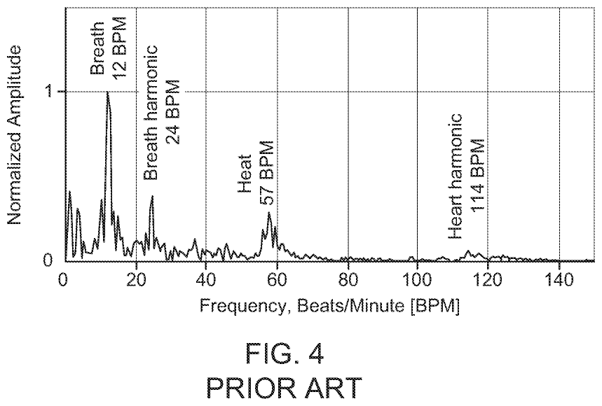

FIG. 4 illustrates a conventional spectrum based method for determining breath rate and heart-rate. The time-domain signal (e.g., time domain signal 308 from FIG. 3) may be transformed using a time-frequency domain transformer into the frequency domain, and the data may be analyzed in the frequency domain as shown in FIG. 4. To convert the time-domain signal into the frequency domain, one of several methods may be used. For example, a Fast Fourier Transform (FFT) may be performed on the signal, where a Hanning window is applied to eliminate frequency leakage and then auto-correlation is applied to make the periodic signals more explicit.

Thereafter, the frequency plot of the signal can be analyzed to determine the heart-rate and breath-rate. Both breathing and heartbeat have different frequencies. Therefore, by checking the frequencies with the highest amplitude in the breathing frequency range and heartbeat frequency range, the respiratory rate and heart rate can be detected. As seen in FIG. 4, the frequency plot can be used to detect the breath rate at 12 breaths/minute and the heart rate at 57 beats/minute.

Other conventional methods of converting the time-domain signal into a frequency plot include applying a Short-Time Fourier Transform (STFT) or a Continuous Wavelet Transform (CWT).

The spectrum-based methods mentioned above, however, rest on an assumption that body vibrations arise only from breathing and heart-beat and are, therefore, susceptible to the same problem as other spectrum techniques. They are not sufficiently accurate because they cannot reliably distinguish between chest region displacements due to heartbeat and respiration from displacement caused by other factors such as random subject movement. Similar to the spectrum-based methods, other methods, including parameter estimation methods, are also incapable of reliably accounting for random subject movements when attempting to detect respiratory rate and heart-beat. Furthermore, both the spectrum-based and parameter-estimation methods are susceptible to other problems including the problem of filtering out noise from other sources such as multi-path reflections, system noise, and irregular breathing and heartbeat.

One of the reasons conventional methods of using continuous-wave radar systems to monitor vital signs are difficult to improve upon is that the approaches used are data independent. In other words, even with the collection of data from several different subjects, the performance of these methods cannot be improved.

Embodiments of the present invention enable contactless detection of at least one of a heart rate and a respiratory rate of a subject using machine learning methods, which can advantageously be trained to be less sensitive to random movements of the subject. Machine learning is the umbrella term for computational techniques that allows models to learn from data rather than following strict programming rules. Machine learning algorithms build a mathematical model based on sample data, known as “training data,” in order to make predictions or decisions without being explicitly programmed to perform the task. Machine learning includes using several different types of models including artificial neural networks (ANNs), decision trees, kernel-based methods, logistic regression.

As discussed above, artificial neural networks (ANN) or connectionist systems are computing systems that are inspired by, but not identical to, biological neural networks that constitute animal brains. Such systems “learn” to perform tasks by considering examples, generally without being programmed with task-specific rules. Neural networks include recurrent neural networks (RNN), convolutional neural networks, and deep belief networks, etc. Some neural networks have multiple layers that enable hierarchical feature learning.

Deep learning (also known as deep structured learning or hierarchical learning) is part of the broader family of machine learning methods based on neural networks. Deep learning describes learning that includes learning hierarchical features from raw input data and leveraging such learned features to make predictions associated with the raw input data.

In particular, embodiments of the present invention train a machine-learning model (e.g., a CNN, a RNN, etc.) to predict the heart-rate and respiratory rate by collecting and using measurements (including, for example, actual heart rate measurements from an electrocardiogram (EKG) monitor) from a variety of test subjects using machine-learning methods, e.g., neural networks and deep-learning methods. Having trained the neural network, embodiments of the present invention can use the neural network to predict the heart-rate and respiratory rate for subjects accurately (without needing additional data from, for example, an EKG monitor). By using a neural network to train over several test subjects, each with their own unique movements, embodiments of the present invention are able to automatically provide significantly more accurate results for new subjects. The trained neural network is able to account for random subject movements based on information cognized through the training process and knowledge embedded in the network.

Embodiments of the present invention are advantageous because they are data-driven. Data collected from test subjects can be used to train the neural network and, accordingly, in this way, the problem is modeled and the noise is minimized.

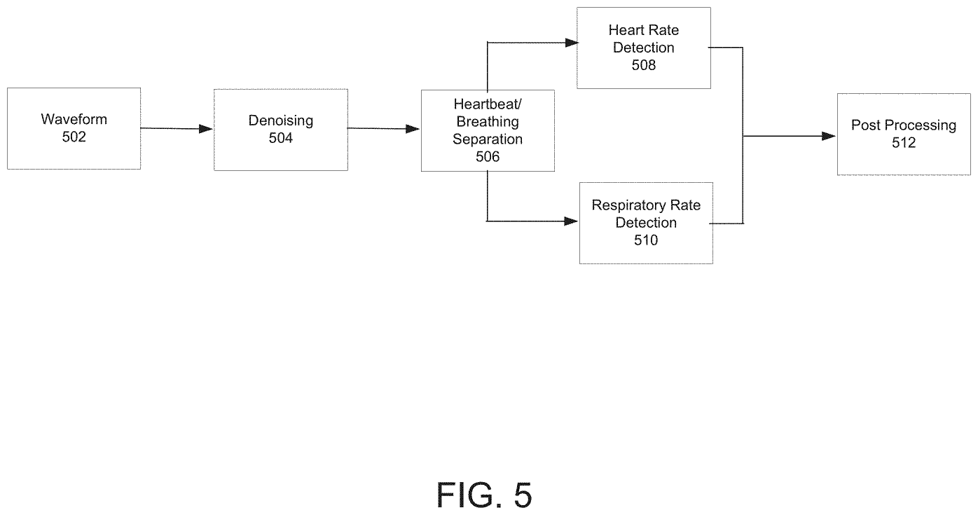

FIG. 5 illustrates a high-level flow diagram providing an overview of the manner in which heart-rate and respiratory rate may be detected in accordance with an embodiment of the present invention. This diagram illustrates data movement and processing blocks that may be implemented by an electronic device.

At block 502, the waveform is unwrapped from the range profile (as discussed in connection with FIG. 3) and received as a time-domain signal. It should be noted that embodiments of the present invention are not limited to FM-CW chirp signals, but also to a wide variety of radar signals including signals obtained, for example, from Impulse Radio Ultra-Wide Band (IR-UWB) radars.

At block 504, denoising is performed on the signal. Several approaches may be used to denoise the signal obtained at block 502. For example, a standardization approach (given by equation f(x)=(x−μ)/σ) may be performed where μ is the mean of the waveform and σ is the standard deviation. This operation also removes any DC component in the signal.

Further, at block 504, a moving average operation may be performed on the signal which aims to remove any sudden changes in the time domain. In addition, at block 504, a Kalman filter may be employed on the signal, which models the velocity of the waveform change in order to compensate for radar body motion.

Finally, at block 504, a band-pass filter may be employed to remove any unwanted frequencies while keeping signals with frequencies in the vital signs range. For example, the respiratory rate can vary between 0.15-0.4 per second while the heart-rate may vary between 0.8 and 2 per second. Accordingly, a band-pass filter may be designed to filter out any frequencies outside of the 0.15 Hz to 2 Hz range. In certain applications, the bandwidth of a band-pass filter may also be increased to 0.15 to 4 Hz to detect any irregularly fast heart-beats or respiratory rates.

At block 506, the signal is conditioned to separate the components related to the heart-beat from the components related to respiratory functioning. At block 508, the heart rate is detected while at block 510, the respiratory rate is detected. Finally, at block 512, post-processing is conducted on the signal, e.g., some harmonics may be identified and removed. As shown in FIG. 4, both the breath-rate and the heart-rate may have certain associated harmonics that may need to be filtered out during post-processing.

Embodiments of the present invention use machine learning models, e.g., neural networks and/or deep learning methods to perform the functions of blocks 506, 508 and 510. These methods can be implemented by electronic device components and/or software.

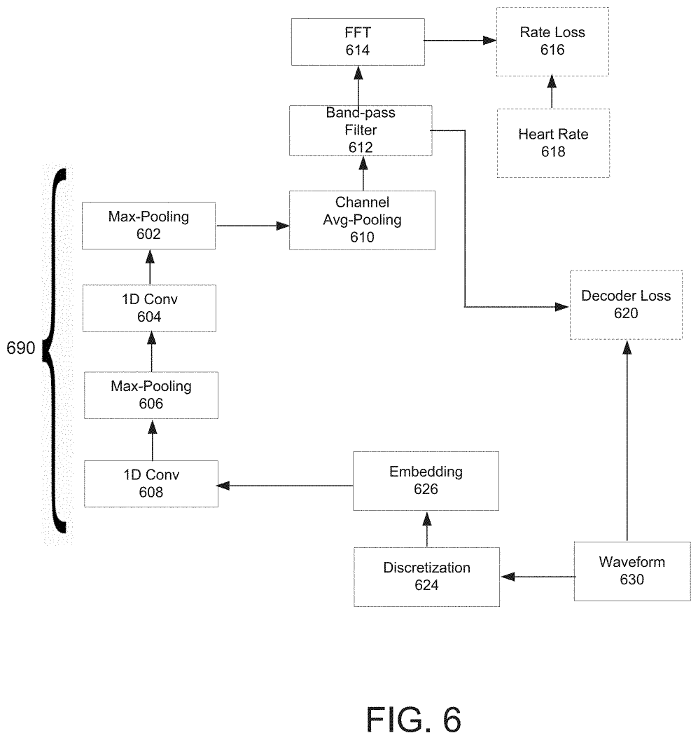

FIG. 6 is a flow diagram indicating the manner in which a machine-learning model such as a neural network may be trained to perform contactless detection of at least one of a heart rate and a respiratory rate of a subject using deep-learning methods in accordance with an embodiment of the present invention.

As noted previously, several different types of neural networks may be used including recurrent neural networks (RNN), convolutional neural networks (CNNs), deep belief networks, etc. RNNs, CNNs and hybrid combinations of RNNs and CNNs (e.g. Deep CNN networks) may have multiple layers that enable hierarchical feature learning. Embodiments of the present invention are not limited to neural networks. Other types of machine learning models may also be used such as decision trees, kernel-based methods, logistic regressions, etc.

FIG. 6 illustrates a two-layer convolution network 690. The network illustrated in FIG. 6, in other words, has two layers of convolution, but may be extended to several layers. Each layer comprises a 1D convolution block (e.g., blocks 604 or 608) with a max-pooling layer (e.g. 602 and 606).

A CNN works well for identifying simple patterns within data (which may then be used to form more complex patterns within higher layers). A 1D CNN is effective for deriving noteworthy features from shorter (fixed-length) segments of the overall data set and where the location of the feature within the segment is not of high relevance. This applies well to the analysis of time sequences of sensor data and to the analysis of any kind of signal data over a fixed-length period (such as time-domain signal 308). Accordingly, the CNN of FIG. 6 uses a 1D convolution block in each layer. It should be noted, however, that the CNN of FIG. 6 may be trained using 2D convolution blocks in each layer as well and is not limited to a 1D convolution block (to apply a 2D convolution block, however, a Short-Time Fourier Transform or a Wavelet Transform may have to be applied to the input waveform)

A pooling layer (e.g. max-pooling layers 602 and 606) is often used after a CNN layer in order to reduce the complexity of the output and prevent over-fitting of the data. For example, choosing a size of 3 for the pooling layer means that the size of the output matrix of this layer is only a third of the input matrix. A max-pooling layer in particular is used to reduce an input size by mapping the size of a given window into a single result by taking the maximum value of the elements in the window.

The waveform for training the neural network is inputted at block 630 of FIG. 6. For example, time-domain signal 308, following the band-pass filtering from the denoising operation at block 504 (in FIG. 5), may be inputted into the neural network. As noted above, the frequency of interest may be between 0.15-4 Hz, so the signal may be filtered using a band-pass filter accordingly. It should be noted that training the neural network requires using waveforms associated with several test subjects. Accordingly, multiple sample waveforms corresponding to several different test subjects need to be used over time to fully train the neural network.

Thereafter, in one embodiment of the present invention, the information is discretized using a discretization module 624. Discretizing performs a binning operation on the real number values corresponding to the waveforms inputted from the waveform block 630. Discretizing continuous features can help improve signal-to-noise ratios. Fitting a model to bins reduces the impact that small fluctuations in the data have on the model because often, small fluctuations are typically simply noise. Each bin smooths out the fluctuations/noises in sections of the data.

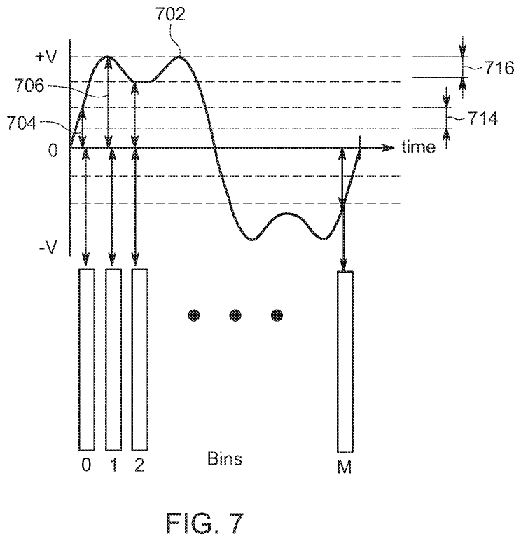

FIG. 7 illustrates an exemplary method in which binning may be performed for the signal waveform containing respiratory rate and heart-rate information in accordance with an embodiment of the present invention. As shown in FIG. 7, the amplitude range of the signal 702 is divided or segmented into sub-ranges, wherein each sub-range corresponds to a discrete bin. For example, signal value 704, occurring within sub-range 714, corresponds to bin 0 while signal value 706, occurring within sub-range 716, corresponds to bin 1. Depending on the sample rate, each discrete value of signal waveform 702 sampled can be assigned to one of the M bins. Each bin may then be represented as an embedding vector.

In one embodiment, embedding may be performed by module 626 in FIG. 6, wherein embedding comprises mapping of a discrete variable to a vector of continuous numbers. Embeddings are low-dimensional, learned continuous vector representations of discrete variables. Neural network embeddings are useful because they can reduce the dimensionality of categorical variables and meaningfully represent categories in the transformed space.

The discretization module 624 and embedding module 626 are useful in the neural network training process because the waveform 630 typically is only a single dimensional vector and directly performing convolution on it may not extract enough information. In other words, performing convolution directly on the waveform 630 results in a model that typically under-fits the data. By first discretizing (or normalizing) the waveform using discretization module 624 into bins and representing each bin as an embedding vector using embedding module 626, different types of noises may be memorized in the embeddings and the model capacity can be increased.

The embedding module 624, as mentioned above, represents each bin as an embedding vector. Accordingly, the bins illustrated in FIG. 6 may be transformed into M vectors (corresponding to the number of bins) of size K dimension, where K depends on the number of values in each bin, which in turn depends on the size of each sub-range (or increment size).

The number of samples collected depends on the window size of the waveform and the longer the window size, the higher the number of samples collected.

Following the discretization-embedding process, convolution operations are performed on the vector data using convolution network 690. As noted above, the network illustrated in FIG. 6, has two layers of convolution, but may be extended to several layers. Each layer comprises a 1D convolution block (e.g., blocks 604 or 608) with a max-pooling layer (e.g. 602 and 606). A convolution is the simple application of a filter to an input that results in an activation. Repeated application of the same filter to an input (e.g., through the use of multiple layers of convolution) results in a map of activations called a feature map, indicating the locations and strength of a detected feature in an input.

More specifically, a convolution is a linear operation that involves the multiplication of a set of weights with the input. For example, a multiplication is performed between a vector of input data and a 1-dimensional array of weights, called a filter or a kernel. The filter is smaller than the input data and the type of multiplication applied between a filter-sized patch of the input and the filter is a dot product. A dot product is the element-wise multiplication between the filter-sized patch of the input and filter, which is then summed, always resulting in a single value. Because it results in a single value, the operation is often referred to as the “scalar product.” Using a filter smaller than the input is intentional as it allows the same filter (set of weights) to be multiplied by the input array multiple times at different points on the input. Specifically, the filter is applied systematically to each overlapping part or filter-sized patch of the input data, left to right, top to bottom.

The max-pooling modules (e.g., modules 602 and 606), as noted above, may be used to reduce the input size by mapping the size of a given window into a single result by taking the maximum value of the elements in the window.

The output of the convolution network 690 may then be directed to a channel average-pooling module 610. The average pooling module 610 is another pooling layer to further avoid over-fitting. This time a value other than the maximum value is taken, namely, the average value of the channels is taken within the neural network. The average-pooling module 610 transforms the matrix output of the convolution network 690 into a single vector. For example, the output of the convolution network may be a set of N vectors (where N is the number of layers of convolution or channels in the network). The average pooling module takes an average between the set of N vectors and transforms the output into a single vector. Other operations that map a matrix to a vector, include the average-pooling, can also be used here

A standard band-pass filter 612 is applied to the vector outputted from channel average-pooling module 610.

Directly modeling the heart-rate by mapping the results of the average pooling module 610 through a multilayer perception (MLP) process typically produces sub-par results because it is difficult to control the network complexity. To account for this, in one embodiment of the present invention, an FFT operation 614 is performed on the band-pass filtered results from block 612 to obtain the frequency distribution.

Subsequently, the softmax loss 616 is calculated against the ground truth heart rate 618. In machine-learning, the term “ground truth” refers to verifiable or actual data that is gathered to train the neural network. The term “ground truthing” refers to the process of gathering the proper objective (verifiable or provable) data for the test. The ground truth heart rate 618 may, for example, be obtained through an EKG monitor and stored in computer-readable memory. The comparison is used to train the neural network.

The discrepancies between the ground truth data 618 and the FFT output 614 of the neural network are determined using the rate loss block 616. The softmax function outputs a vector that represents the probability distributions of a list of potential outcomes. A softmax activation will typically take the vector from FFT block 614 and reduce the vector down using another matrix multiplication. The softmax is used as an activation function that takes the FFT outputs of the neural network and forces them to sum up to one.

The output of the softmax module 616 is compared against the ground truth heart rate 618 to determine where the discrepancies (or losses) are. In other words, the comparison is used to determine where the output of the neural network data is deviating from the norm.

It should be noted that both the soft-max loss block 616 and the ground truth heart rate 618 are only required during the training of the neural network. The computed loss is used for gradient calculation and updating network parameters through back-propagation. Back-propagation is a way of propagating the total loss back into the neural network to account for the degree of the loss every node is responsible for, and subsequently updating the weights to minimize the loss by assigning the nodes with higher error rates lower weights and vice versa. Back-propagation is a critical element of neural net training. It is the practice of fine-tuning the weights of a neural net based on the error rate (e.g., loss) obtained in the previous epoch (e.g., iteration). Proper tuning of the weights ensures lower error rates, making the model reliable by increasing its generalization.

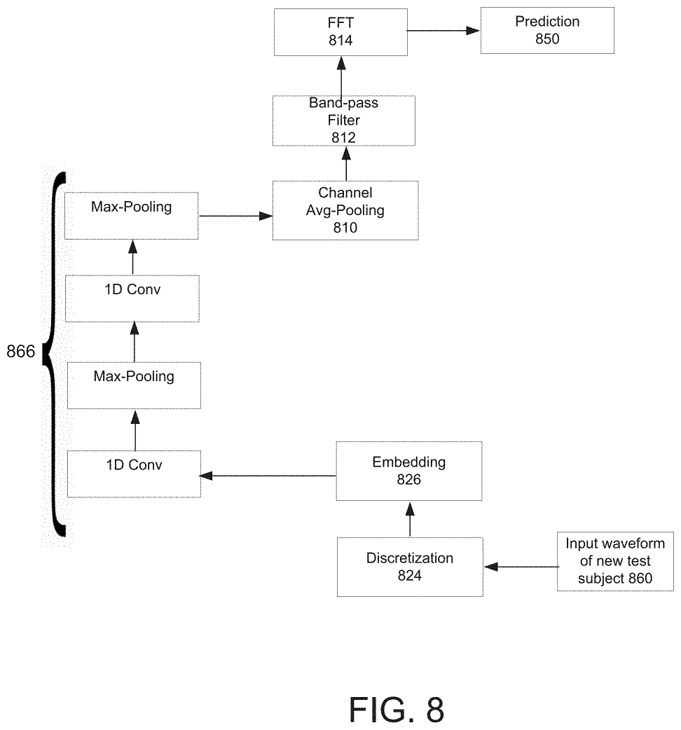

The blocks pertaining to back-propagation (specifically blocks 616, 618 and 620 in FIG. 6) are only used in association with training the network. When the network is used to make actual predictions for heart-rate and respiratory rate, blocks 616 and 618 are no longer required (as will be explained in further detail in connection with FIG. 8).

Additional denoising is also performed by comparing the output of the bandpass filter 612 with the input waveform 630 using decoder loss block 620. The decoder loss comprises computing the difference between the original waveform (ground truth data) and the constructed waveform from the neural network model using mean square loss. Decoder loss block 620 is also used to train the neural network by computing gradients for a parameter update (using back propagation) and, therefore, is not required when performing actual predictions related to heart-rate and respiratory rate.

FIG. 8 is a flow diagram indicating the manner in which a machine-learning model such as neural network may be used to perform contactless prediction of a heart-rate and a respiratory rate using deep-learning methods in accordance with an embodiment of the present invention.

FIG. 8 follows closely from FIG. 6 but does not comprise blocks 618, 618 and 620 that are used to train the neural network. The input data for a new test subject (whose vital signs need to be detected) are inputted as a (slow time) time-domain signal at block 860. Discretization and embedding are performed by blocks 824 and 826 respectively. Subsequently, a number of vectors (equal to the number of channels) are derived from the data by performing convolution and max-pooling using the convolution network 866. The vectors are thereafter consolidated into a single vector using the channel average-pooling block 810. A band-pass filter operation 812 and an FFT operation 814 is performed on the vector to yield a prediction for a heart-rate or a respiratory rate 850. This data may be stored in computer memory. In one embodiment, post-processing may be conducted on the predictions to remove any influence pertaining to the harmonics of a heart-rate or respiratory rate.

In one embodiment, the neural network of FIG. 6 may be first trained on a respiratory rate and the body movements related thereto. Subsequently, using information pertaining to respiratory rate and the body movements related thereto, the network could be trained again to use the information to predict a heart-rate. Thereafter, the neural network illustrated in FIG. 8 may be used to predict a heart-rate using the previously learned information pertaining to respiratory rate and the subject's associated body movements.

In one embodiment, more accurate respiratory rate and heart rate information can be predicted for a particular subject by allowing the neural network to first train on the subject. In other words, the neural network is customized by training on the subject. In other words, ground truth data for a specific subject may be used to first update the neural network. Subsequently, more accurate contactless measurements for the user may be predicted because training data specific to the subject is incorporated into the model. In a different embodiment, a standard model (not including data specific to the subject) may also be used to predict heart rate and respiratory rate for the individual, but it may not be as accurate as allowing the neural network to train with the data from the specific subject.

Embodiments of the present invention provide results that are significantly more accurate than prior radar-based methods of detecting heart-rate and respiratory rate. For example, embodiments of the present invention can provide accuracy of within 4 heart-beats/minute whereas prior methods only provide an accuracy of within at most 10 heart-beats/minute.

Embodiments of the present invention may be used in a wide variety of applications. For example, systems comprising embodiments of the present invention may be installed at airports and used to screen passengers non-invasively for infectious diseases using heart-rates and respiratory rates. Embodiments of the present invention may also be installed in cars and used to detect a driver's vital signs and provide warnings to the driver if their vital signs go below a certain threshold. Additionally, embodiments of the present invention can also be used to reliably detect physiological conditions like the flu in large groups of humans and animals by monitoring their vital signs non-invasively.

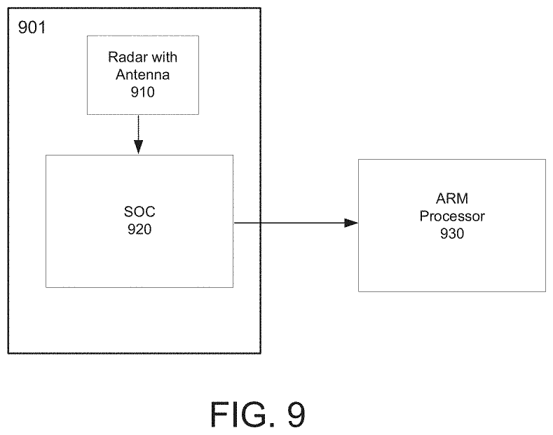

FIG. 9 illustrates an exemplary electronic device apparatus for performing contactless prediction of a heart-rate and respiratory rate using a machine-learning model in accordance with an embodiment of the present invention.

An electronic circuit board 901 comprises an on-chip radar sensor 910 with an antenna for receiving a signal, for example, from a test subject. The radar signal is transmitted to a System-On-Chip (SOC). The SOC may comprise, for example, a digital signal processor (DSP) with a micro-controller unit (MCU) and memory. The SOC converts the radar signal into a waveform in the time-domain (e.g. signal 308).

The time-domain signal is then transmitted to a microprocessor, e.g., an ARM processor. The microprocessor is typically programmed to train the neural network (as described in conjunction with FIG. 6) and to use the neural network to perform predictions (as described in conjunction with FIG. 8). Further, the microprocessor may also be programmed to conduct post-processing as discussed in connection with FIG. 5.

It should be noted that the electronic device apparatus in FIG. 9 is exemplary and that embodiments of the present invention may be practiced using a wide variety of processing components.

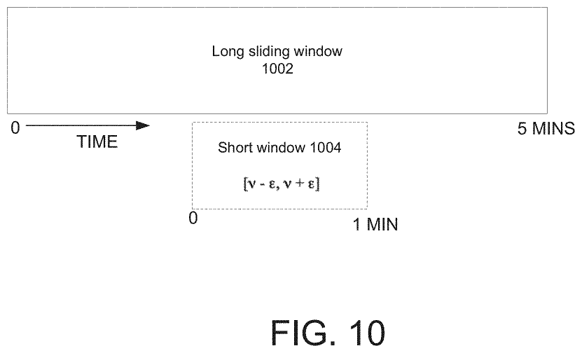

FIG. 10 illustrates the manner in which a long sliding time window may be maintained in conjunction with a short sliding window in order to detect sudden changes in heart-rate in accordance with an embodiment of the present invention.

One of the challenges of using long sliding time windows when analyzing the radar signals from a test subject is that any sudden changes in heartbeat may go undetected. For example, if the time-domain signal (e.g. signal 308 in FIG. 3 or signal 702 in FIG. 7) analyzed comprises longer time-windows (e.g., 5 minutes or more), then it is possible that the neural network may not detect sudden temporary changes in heartbeat. If the detected heart rate changes significantly, it may be caused, for example, by cross-range-bin movements as a result of body motion. Any type of radar detection system would need time to detect and fix this issue. However, the motion may only be for a short period of time and it is important to capture this data.

In one embodiment, this problem may be accounted for by maintaining a shorter sliding time-window of analysis 1004 in conjunction with the longer sliding window 1002. Further, it is assumed that over a short period of time the human heart-rate will not change significantly, but, in fact, will only diverge within a narrow frequency range.

The long sliding window 1002 (which may, for example, be 5 minutes long) may be used to estimate a rough heart rate over the entire duration. Thereafter, around the time range (within the long window) where the heartbeat is irregular, the data is filtered in a short sliding window (which may, for example, be a 1 minute long) to a narrow frequency range. Assuming, the rough heart-rate detected using the long sliding window is ν, then the narrow frequency range may be represented by [ν−ε, ν+ε] where ε is the confidence parameter. For example, if the long sliding window detects an approximate heart rate of 1.2, the short sliding window is used assuming that the heart-rate will not vary more than, for example, 0.2 Hz from 1.2, where 0.2 is the confidence parameter. Accordingly, any frequencies less than 1.0 (1.2−0.2) or greater than 1.4 (1.2+0.2) may be band-pass filtered. Filtering out the other frequencies effectively removes the interference caused by random body motions leaving only frequencies that are most likely associated with the actual aberrant heart-rate. Thereafter, the irregular heart-beat within the short sliding window can be estimated using the filtered data.

In one example, the approximate heart rate using the long sliding window may be estimated using the neural network of FIG. 8. Subsequently, the user may want to determine an irregular heart beat of a test subject within a short sliding time range within the long sliding window. By band-pass filtering the frequencies in the narrow sliding window using the approximate heart rate information gathered from FIG. 8, an estimate of the irregular heart beat in the narrow sliding window can be derived using embodiments of the present invention.

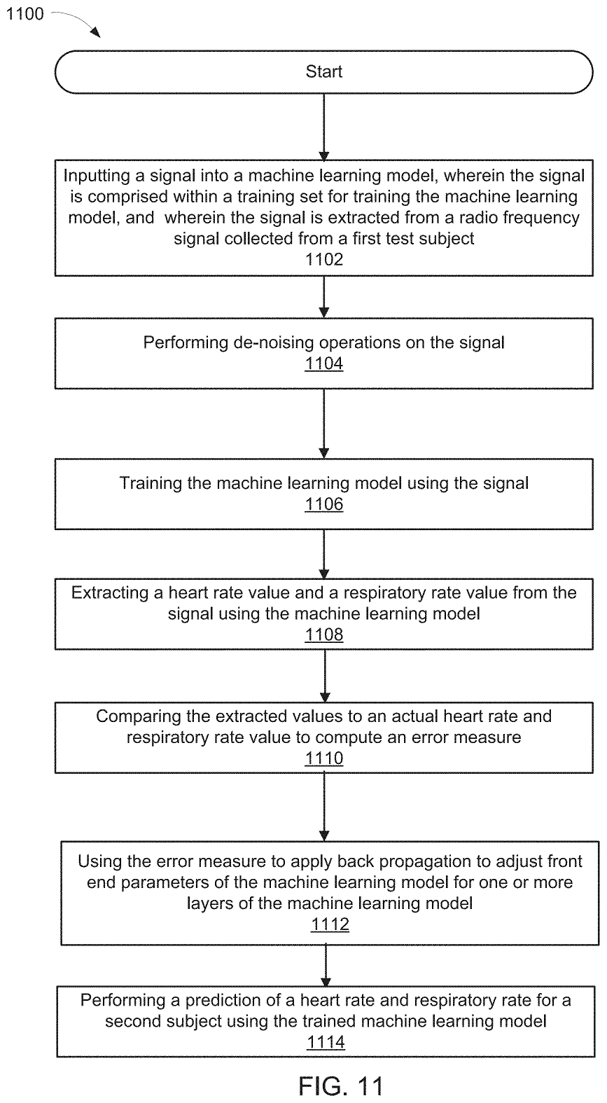

FIG. 11 depicts a flowchart illustrating an exemplary computer-implemented process for training and using a machine learning model (e.g. a neural network) to detect respiration rate and heart rate in accordance with an embodiment of the present invention. While the various steps in this flowchart are presented and described sequentially, one of ordinary skill will appreciate that some or all of the steps can be executed in different orders and some or all of the steps can be executed in parallel. Further, in one or more embodiments of the invention, one or more of the steps described below can be omitted, repeated, and/or performed in a different order. Accordingly, the specific arrangement of steps shown in FIG. 11 should not be construed as limiting the scope of the invention. Rather, it will be apparent to persons skilled in the relevant art(s) from the teachings provided herein that other functional flows are within the scope and spirit of the present invention. Flowchart 1100 may be described with continued reference to exemplary embodiments described above, though the method is not limited to those embodiments.

At step 1102, a signal (e.g. time-domain signal 308 from FIG. 3) is inputted into a machine learning model, e.g., a neural network, where the signal is comprised within a training set for training the neural network. As mentioned above, embodiments of the present invention may be able to use any one of several types of machine learning models, including neural networks, recurrent neural networks (RNN), convolutional neural networks (CNNs), deep belief networks. Deep Learning models, decision trees, kernel-based methods, logistic regression, etc.

The time-domain signal 308 may be derived from a signal from an FM-CW radar system or other types of radar systems including bio-radar. The time-domain signal is extracted from the radio frequency signal reflected by the moving tissue of a first test subject. As noted above, in the receiver, the signal transmitted from the radar system may be mixed with the reflected Doppler-shifted signal (from the test subject) to produce a mixing product which, following low pass filtering, results in a baseband signal including a low frequency component that is directly proportional to the instantaneous surface displacement of the tissue of the first test subject.

At step 1104, de-noising operations are performed on the signal as explained in connection with block 504 of FIG. 5.

At step 1106 of FIG. 11, the signal is used to train the machine learning model, e.g., a neural network through a combination of I-D convolution operations, max-pooling and channel average-pooling as explained in connection with FIG. 6. As noted previously, the machine learning model, e.g., a neural network may comprise one or several layers of convolution.

At step 1108, a heart rate and respiratory rate are extracted from the signal. In one embodiment, a band-pass filtering operation (e.g., using block 612 of FIG. 6) and an FFT operation (e.g., using FFT block 614 of FIG. 6) is performed on the signal before the heart rate and respiratory rate can be extracted.

At step 1110, the extracted values are then compared to ground truth data to compute an error measure. For example, as discussed in connection with FIG. 6, discrepancies between the ground truth data 618 and the FFT output 614 of the neural network are determined using the rate loss block 616. As also noted in the discussion above, additional denoising is also performed by comparing the output of the bandpass filter 612 with the input waveform 630 using decoder loss block 620.

At step 1112, the error measure is used to apply back propagation to adjust front end parameters of the machine learning model for one or more layers of the neural network. The back propagation effectively trains the machine learning model to improve its predictions using the information from the first test subject.

At step 1114, an input signal corresponding with a second test subject may be inputted into the trained machine learning model, which will then predict a heart rate and respiratory rate for the second test subject.

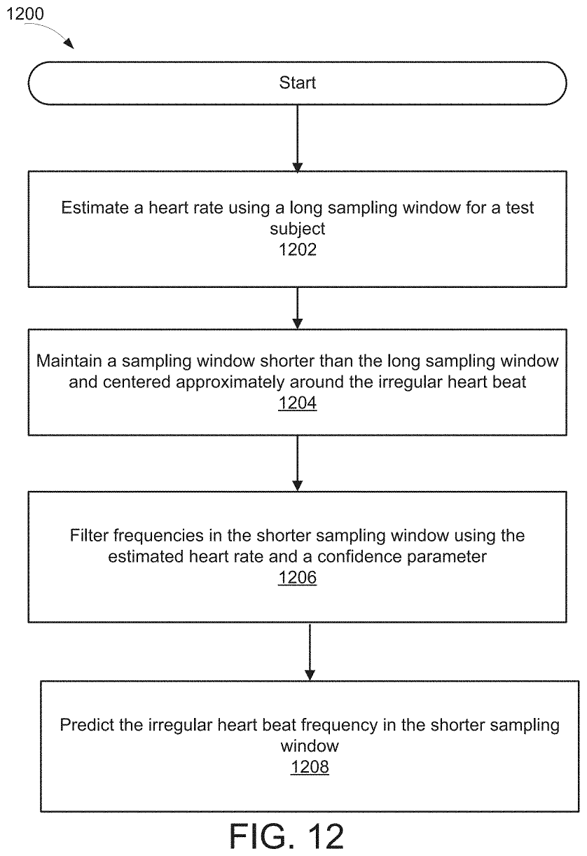

FIG. 12 depicts a flowchart illustrating an exemplary computer-implemented process for using a machine learning model to detect sudden changes in heart rate in accordance with an embodiment of the present invention. While the various steps in this flowchart are presented and described sequentially, one of ordinary skill will appreciate that some or all of the steps can be executed in different orders and some or all of the steps can be executed in parallel. Further, in one or more embodiments of the invention, one or more of the steps described below can be omitted, repeated, and/or performed in a different order. Accordingly, the specific arrangement of steps shown in FIG. 12 should not be construed as limiting the scope of the invention. Rather, it will be apparent to persons skilled in the relevant art(s) from the teachings provided herein that other functional flows are within the scope and spirit of the present invention. Flowchart 1200 may be described with continued reference to exemplary embodiments described above, though the method is not limited to those embodiments.

As discussed in connection with FIG. 10, one of the challenges of using long period of time sliding time windows when analyzing the radar signals from a test subject is that any sudden or irregular changes in heartbeat may go undetected. Embodiments of the present invention address this issue by maintaining a shorter sliding time-window of analysis 1004 in conjunction with the longer sliding window 1002. Further, it is assumed that over a short period of time the human heart-rate will not change significantly, but, in fact, will only diverge within a narrow frequency range.

Accordingly, at step 1202 a heart rate is estimated for a test subject using a long sampling window. The estimated heart rate may, for example, be obtained using a neural network as discussed in connection with FIG. 8 (or by any other methods).

At step 1204 of FIG. 12, a shorter sliding time-window of analysis 1004 is maintained in conjunction with the longer sliding window 1002, where the shorter sliding window is centered approximately around the region of interest, e.g., around the occurrence of the irregular heart-beat.

At step 1206, the data in the shorter sliding window is filtered to a narrow frequency range. Assuming, the rough heart-rate detected using the long sliding window is ν, then the narrow frequency range may be represented by [ν−ε, ν+ε] where ε is the confidence parameter. Any frequencies outside of the [ν−ε, ν+ε] range are filtered out. Filtering out the other frequencies effectively removes the interference caused by random body motions leaving only frequencies that are most likely associated with the actual aberrant heart-rate. Thereafter, at step 1208, the irregular heart-beat within the short sliding window can be estimated using the filtered data.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the invention and its practical applications, to thereby enable others skilled in the art to best utilize the invention and various embodiments with various modifications as may be suited to the particular use contemplated.

What is claimed is:

1. A computer-implemented method of determining a heart rate and respiratory rate from a radio frequency signal, the method comprising:
inputting a first radio frequency signal representing a first test subject into a machine-learning model, wherein the first radio frequency signal is comprised within a training set for training the machine-learning model;
training the machine-learning model using the first radio frequency signal;
determining a first heart rate and a first respiratory rate from the first radio frequency signal using the machine-learning model;
computing an error measure by comparing the first heart rate and the first respiratory rate determined from the first radio frequency signal to a verifiable heart rate and verifiable respiratory rate for the first test subject; and
improving a prediction accuracy of the machine-learning model by using the error measure to apply back propagation to adjust front end parameters for one or more layers of the machine-learning model.

2. The computer-implemented method of claim 1, further comprising:
applying the machine-learning model to a second radio frequency signal representing a second test subject to predict a second heart rate and second respiratory rate for the second test subject, wherein values of the second heart rate and the second respiratory rate are more accurate than the first heart rate and the first respiratory rate.

3. The computer-implemented method of claim 1, further comprising, prior to the inputting:
converting the first radio-frequency signal into a time-domain signal; and
performing a de-noising operation on the time-domain signal.

4. The computer-implemented method of claim 1, wherein the machine-learning model is of a type selected from a group consisting of: an artificial neural network; a recurrent neural network (RNN); a convolutional neural network (CNNs); a deep belief network; a Deep Learning network; a decision tree; a kernel-based method, and a logistic regression; and a combination of one or more machine-learning models.

5. The computer-implemented method of claim 1, wherein the machine-learning model comprises a convolutional neural network comprising one or more layers, and wherein each layer comprises a 1-D convolution layer and a max-pooling layer.

6. The computer-implemented method of claim 6, wherein an output of the one or more layers is averaged using an average-pooling layer.

7. The computer-implemented method of claim 3, wherein the training comprises:
discretizing the time-domain signal;
representing an output of the discretizing as one or more embedding vectors;
performing convolution and max-pooling operations on the one or more embedding vectors using one or more layers of the machine-learning model; and
performing a channel average-pooling operation on an output of the one or more layers of the machine-learning model to yield a single vector with average values.

8. The computer-implemented method of claim 7, further comprising, prior to the comparing:
performing a band-pass filter operation on values in the single vector outputted from the average-pooling operation; and wherein the
determining the first heart rate and the first respiratory rate comprises performing a Fast Fourier Transform (FFT) operation on the values in the single vector subsequent to the band-pass filter.

9. The computer-implemented method of claim 8, further comprising:
comparing an output of the band-pass filter operation with the first radio frequency signal to determine a decoder loss; and
using the decoder loss to apply back propagation to adjust the front end parameters for the one or more layers of the machine-learning model to reduce noise in results obtained from the machine-learning model.

10. A non-transitory computer-readable storage medium having stored thereon, computer executable instructions that, if executed by a computer system cause the computer system to perform a method of determining a heart rate from a wireless signal, the method comprising:
inputting a first wireless signal obtained from a first test subject into a machine-learning model, wherein the first wireless signal is comprised within a training set for training the machine-learning model;
training the machine-learning model using the first wireless signal;
extracting a first heart rate from the first wireless signal using the machine-learning model;
comparing the first heart rate extracted from the wireless signal to a verifiable heart rate for the first test subject to compute an error measure; and
using the error measure to apply back propagation to adjust front end parameters for one or more layers of the machine-learning model to improve a prediction accuracy of the machine-learning model.

11. The non-transitory computer-readable storage medium of claim 10, wherein the method further comprises:
applying the machine-learning model to a second wireless signal obtained from a second test subject to predict a second heart rate for the second test subject, wherein a value of the second heart rate is more accurate than the first heart rate.

12. The non-transitory computer-readable storage medium of claim 10, wherein the method further comprises, prior to the inputting:
converting the wireless signal into a time-domain signal.

13. The non-transitory computer-readable storage medium of claim 10, wherein the machine-learning model is a convolutional neural network comprising one or more layers, and wherein each layer comprises a 1-D convolution layer.

14. The non-transitory computer-readable storage medium of claim 10, wherein the wireless signal is a Frequency-modulated continuous-wave (FM-CW) radar signal.

15. The non-transitory computer-readable storage medium of claim 12, wherein the training comprises:
discretizing the time-domain signal;
representing an output of the discretizing as one or more embedding vectors;
performing convolution and max-pooling operations on the one or more embedding vectors using one or more layers of the machine-learning model; and
performing a channel average-pooling operation on an output of the one or more layers of the machine-learning model.

16. A system for determining a respiratory rate from a radio frequency signal, the system comprising:
a memory for storing a time-domain representation of one or more radio frequency signals, instructions associated with a neural network and a process of determining the respiratory rate from the radio frequency signal;
a processor coupled to the memory, the processor configured to operate in accordance with the instructions to:
input a first radio frequency signal obtained from a first test subject into the neural network, wherein the first radio frequency signal is comprised within a training set for training the neural network;
train the neural network using the first radio frequency signal;
extract a first respiratory rate from the first radio frequency signal using the neural network;
compare the first respiratory rate determined from the first radio frequency signal to a verifiable respiratory rate for the first test subject to compute an error measure; and
use the error measure to apply back propagation to adjust front end parameters for one or more layers of the neural networks to improve a prediction accuracy of the neural network.

17. The system of claim 16, wherein the processor is further configured to:
apply the neural network to a second radio frequency signal obtained from a second test subject to predict a second respiratory for the second test subject, wherein values of the second respiratory is more accurate than the first respiratory rate.

18. The system of claim 16, wherein the processor is an ARM processor.

19. The system of claim 16, wherein the processor is further configured to:
perform post-processing on the second respiratory rate to filter out an influence of harmonics associated with the second respiratory rate.

20. The system of claim 16, wherein the neural network is a convolutional neural network comprising one or more layers, wherein each layer comprises a 2-D convolution layer.

21. The system of claim 16, wherein the neural network is of a type selected from a group consisting of: a recurrent neural network (RNN); a convolutional neural network (CNNs); a deep belief network; and a Deep Learning network.
